(19) United States
(12) Patent Application Publication
(10) Pub. No.: US 2022/0152241 A1
(43) Pub. Date: May 19, 2022

(54) SANITIZED FINGERPRINT SCANNER

(71) Applicant: TECHNOBRAVO LLC, Oakland, NJ (US)

(72) Inventors: Warren Lahoud, Oakland, NJ (US); Charles Laxton, Oakland, NJ (US); Darren Reid, Oakland, NJ (US)

(21) Appl. No.: 17/527,053

(22) Filed: Nov. 15, 2021

Related U.S. Application Data
(60) Provisional application No. 63/113,933, filed on Nov. 15, 2020.

(51) Int. Cl. A61L 2/10; G06K 9/00; A61L 2/26

(52) U.S. Cl. CPC A61L 2/10; A61L 2202/11; A61L 2/26; G06K 9/00013

(57) ABSTRACT

Systems, devices, and methods for sanitizing a fingerprint scanner include a sanitized fingerprint scanner comprising a housing, one or more signaling lights, one or more sensors, one or more UV light arrays, and a fingerprint scanner having a fingerprint scanner surface, the sanitized fingerprint scanner configured to: obtain one or more fingerprints of a first user at the fingerprint scanner surface; after obtaining the one or more fingerprints of the first user, enabling the one or more UV light arrays to radiate light toward the fingerprint scanner surface, and after radiating the UV light toward the fingerprint scanner surface for a predetermined period of time, authorizing a second user to use the fingerprint scanner.

### PRIORITY INFORMATION

This application is a non-provisional application that claims the benefit of U.S. Provisional Application No. 63/113,933, filed on Nov. 15, 2020, the contents of which are herein incorporated by reference in their entirety.

### BACKGROUND OF THE PRESENT INVENTION

### 1. Field of the Present Invention

The embodiments of the present invention generally relate to a fingerprint scanner, and more particularly, to a fingerprint scanner that includes ultraviolet (UV) lighting to sanitize a fingerprint contact surface.

### 2. Description of the Related Art

Currently, several biometric and other electronic devices are used in connection with an increasing demand for accurate personal identification. Generally, biometric devices use fingerprints, eye retinas, eye irises, voices, facial patterns, electronic signatures, and the like to identify a user.

While several types of biometric devices can be used for personal identification, fingerprint scanners remains among the most common. Now, in view of the Covid-19 pandemic and possible future pandemics, there will be a reluctance to continue using fingerprint scanners because a single surface is contacted by many users, and there is a likelihood of spreading viruses, bacteria, and other contagions. In other words, fingerprint scanners will no longer be viewed as merely non-invasive and harmless during and after the Covid pandemic.

The typical approach is for users to manually sanitize their hands with a hand sanitizer or other detergent before/after use of the fingerprint scanner. Alternatively, current sanitization techniques of the biometric fingerprint reader surfaces include the manual wiping with a fungicide, bactericide, and/or virucide after and/or before each use by a respective user.

Accordingly, many related art fingerprint scanners are unhygienic because of the shared use of the fingerprint contact surface. In view of these drawbacks and the widespread use of fingerprint scanners, the inventors have developed systems, devices, and methods for sanitizing existing (e.g., currently deployed or commercially available) fingerprint scanners, and avoiding the cost of their complete replacement. In addition, the embodiments developed by the inventors do not require any prior and/or post use wipe-down or prior and/or post use hand sanitizers or detergent.

### SUMMARY OF THE PRESENT INVENTION

Accordingly, the embodiments of the present invention are directed to systems, devices, and methods for sanitizing a fingerprint scanner or other electronic device that substantially obviate one or more problems due to limitations and disadvantages of the related art.

An object of the present invention is to provide a fingerprint scanner or other electronic device used by a plurality of users in order to add a sterilizing function thereto and improve hygienic conditions thereof.

Additional features and advantages of the present invention will be set forth in the description which follows, and in part will be apparent from the description, or can be learned by practice of the present invention. The objectives and other advantages of the present invention will be realized and attained by the structure particularly pointed out in the written description and claims hereof as well as the appended drawings.

To achieve these and other advantages and in accordance with the purpose of the present invention, as embodied and broadly described, the systems, devices, and methods for sanitizing a fingerprint scanner include a sanitized fingerprint scanner comprising a housing, one or more signaling lights, one or more sensors, one or more UV light arrays, and a fingerprint scanner having a fingerprint scanner surface, the sanitized fingerprint scanner configured to: obtain one or more fingerprints of a first user at the fingerprint scanner surface; after obtaining the one or more fingerprints of the first user, enabling the one or more UV light arrays to radiate light toward the fingerprint scanner surface, and after radiating the UV light toward the fingerprint scanner surface for a predetermined period of time, authorizing a second user to use the fingerprint scanner.

It is to be understood that both the foregoing general description and the following detailed description are exemplary and explanatory and are intended to provide further explanation of the present invention as claimed.

### BRIEF DESCRIPTION OF THE DRAWINGS

The accompanying drawings, which are included to provide a further understanding of the present invention and are incorporated in and constitute a part of this application, illustrate embodiments of the present invention and together with the description serve to explain the principle of the present invention.

FIG. 1 illustrates a perspective front view (1A) and perspective rear view (1B) of a sanitized fingerprint scanner according to an example embodiment of the present invention.

FIG. 2 illustrates a perspective semi-transparent front view of a sanitized fingerprint scanner according to an example embodiment of the present invention.

FIG. 3 illustrates an expanded perspective view of a sanitized fingerprint scanner according to an example embodiment of the present invention.

FIG. 4 illustrates a perspective front view of a sanitized fingerprint scanner according to another example embodiment of the present invention.

FIGS. 5, 6, 7 and 8 each illustrate a sanitized kiosk according to example embodiments of the present invention.

FIG. 9 illustrate a sanitized mobile computing device according to an example embodiment of the present invention.

### DETAILED DESCRIPTION OF THE PRESENT INVENTION

Reference will now be made in detail to the embodiments of the present invention, examples of which are illustrated in the accompanying drawings. Wherever possible, like reference numbers will be used for like elements.

Embodiments of the present invention include a variety of user interfaces and associated methods, and are described herein. Although a fingerprint scanner is described as an example embodiment, it should be understood that the embodiments can be readily applied to single-fingerprint scanners, dual-fingerprint scanners, multi-fingerprint scanners (e.g., 4-4-2 fingerprint sensors/readers), palm readers, automated teller machines (ATMs), point of sale systems, point of service systems, pin/keypad devices, kiosks, point of service kiosks, point of sale kiosks, self-service kiosks, gambling machines, border-entry systems, gate-entry systems, access-control systems (e.g., doors, elevators, escalators), etc. In some embodiments, the electronic device is a fixed (e.g., stationary) or portable (e.g., handheld), and the user interface can include a touchscreen and/or other input/output devices (e.g., a keypad, mouse, button, trackpad, knob, etc.). In the discussion that follows, a fingerprint scanner is used as an example embodiment. It should be readily understood, however, that the user interfaces and associated methods can be applied to other devices.

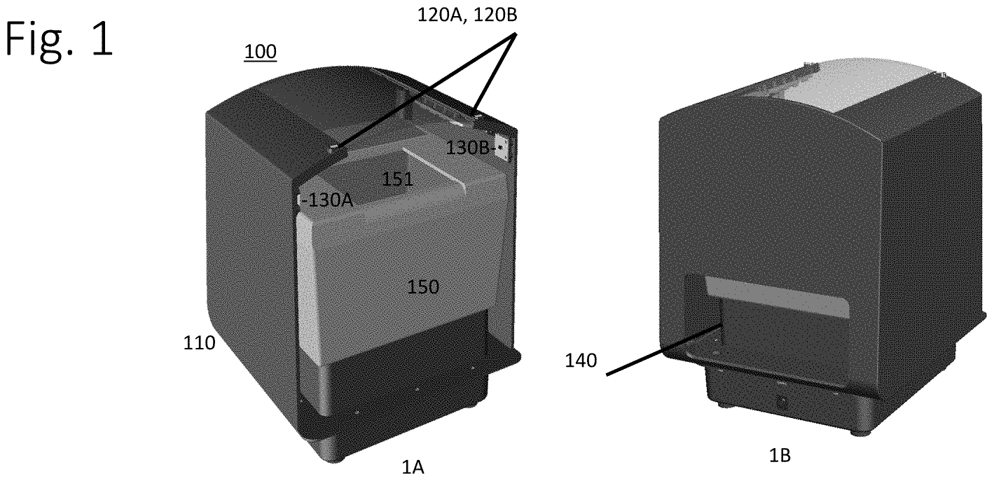

FIG. 1 illustrates a perspective front view (1A) and perspective rear view (1B) of a sanitized fingerprint scanner 100 according to an example embodiment of the present invention. As shown in FIG. 1, sanitized fingerprint scanner 100 includes housing 110, signaling lights 120A, 120B, sensors 130A, 130B, rear opening 140, fingerprint scanner 150, and fingerprint scanner surface 151.

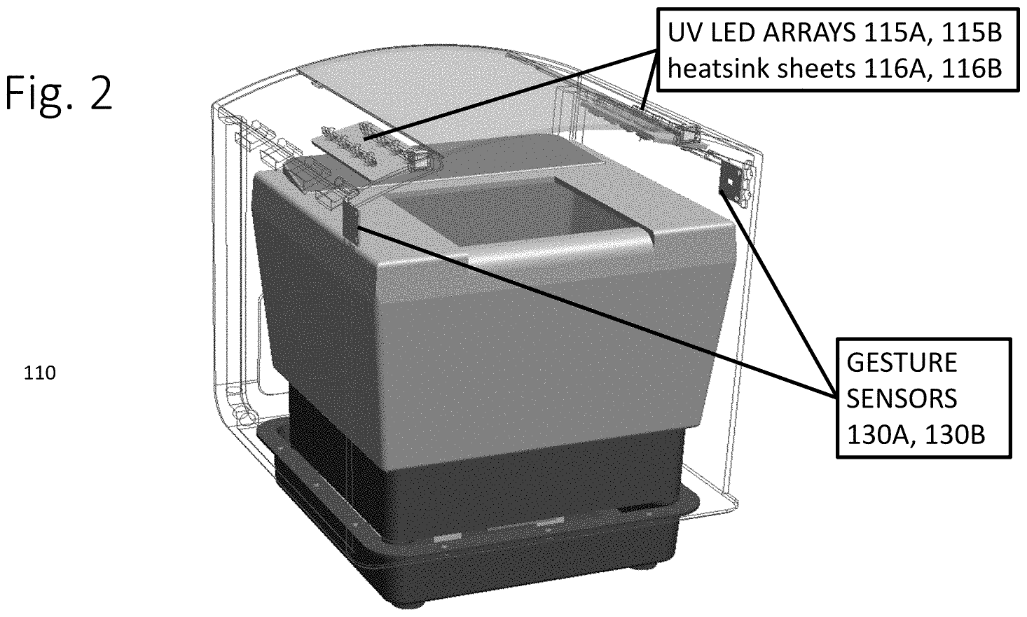

Housing 110 is configured to structurally support the components of the sanitized fingerprint scanner 100 including UV LED arrays 115A, 115B (as shown in FIG. 2), signaling lights 120A, 120B, sensors 130A, 130B, fingerprint scanner 150, etc. Housing 110 can be formed from various lightweight materials. Example materials include a variety of plastics, preferably durable and lightweight plastics (e.g., acrylonitrile butadiene styrene (ABS), poly carbonate (PC), polyvinyl chloride (PVC), or combination/blend thereof). Lightweight metals, such as aluminum, also can be used. Housing 110 can be formed by a molding, injection molding, three-dimensional (3D) printing process.

Signaling lights 120A, 1208 can be low-power LED lights configured to signal and instruct a user to input one or more fingers on the fingerprint scanner surface 151 or to remain clear of the sanitized fingerprint scanner 100 during a UV sanitization timeperiod or process. For example, a green light may be used to authorize a user to insert on or more fingers, and a red light may be used to instruct a user to stand back (e.g., during a UV sanitization process or period).

Sensors 130A, 1308 are configured to detect whether one or more fingers of a user accesses fingerprint contact surface 151. The sensors 130A, 1308 can be a photo-sensor having a light emitting part and a light receiving part, for example. Sensors 130A, 1308 can be disposed around the fingerprint contact surface 151 to detect whether one or more fingers of a user or other object approaches or covers the fingerprint contact surface 151. Although two sensors 130A, 1308 are illustrated, the embodiments are not so limited. One or more sensors of a variety of sensor types can be used.

Sensors 130A, 1308 detect motion to determine if a finger(s) are disposed on the fingerprint contact surface 151 or under the UV lights to prevent exposure of UV light to the user's skin and eyes. Sensors 130A, 1308 are configured to halt or terminate any ongoing UV sanitizing process upon detection of a user's hand, finger, or other object. Other sensors (not shown) can be used to disable the UV sanitizing process when a user is within close proximity (e.g., 3-4 feet) to sanitized fingerprint scanner 100.

Opening 140 is a wide opening on the rear of sanitized fingerprint scanner 100. Opening 140 is configured as a wide opening relative to sanitized fingerprint scanner 100 because of the variety of rear connector types and locations of commercially available fingerprint scanners.

FIG. 2 illustrates a perspective semi-transparent front view of a sanitized fingerprint scanner 100 according to an example embodiment of the present invention. As shown in FIG. 2, sanitized fingerprint scanner 100 includes UV LED arrays 115A, 115B and heat sink sheets 116A, 116B. In this example configuration, heat sink sheets 116A, 116B are disposed between UV LED arrays 115A, 115B and housing 110.

UV LED arrays 115A, 115B are configured to radiate light to the entire fingerprint contact surface 151. UV LED arrays 115A, 115B also can be configured to radiate light to the surroundings of entire fingerprint contact surface 151 that may be touched by users. After the user's one or more fingers is removed from fingerprint contact surface 151, UV LED arrays 115A, 115B are configured to emit UV light to sanitize the fingerprint contact surface 151.

The emitted UV light can be UVA (315-400 nm), UVB (280-315 nm), UVC (100-280 nm, including far-UVC 207-222 nm), or a various combination thereof. In addition, the emitted UV light can be continuous for a predetermined period of time (e.g., 6-7 seconds), or pulsated for a predetermined period of time (e.g., 6-7 seconds). In some configurations, UVC light having a wavelength of 275 nm is used. In other configurations, xenon-based or mercury based UV globes and LED's having an electromagnetic radiation spectrum of between 200 nm and 400 nm can be used. In some configurations, the predetermined period of time and/or the power level of LED arrays 115A, 115B can be varied or set by an operator of sanitized fingerprint scanner 100.

Each of UV LED arrays 115A, 115B can be coupled to an inner surface of housing 110 along one or more LED boards (e.g., PCB). For example, UVA, UVB, UVC, and/or far-UVC lights can be mounted, applied, and/or electrically engaged either individually or in combination under a light housing hood as shown by FIG. 2. Each of UV LED arrays 115A, 115B can contain a multiple (e.g., 8) bulbs to emit UV light. The inclination angle of the radiated UV light may be determined by the light housing hood or other electrical or mechanical means. Each of the bulbs can be configured to emit either a single type of UV light or multiple types of UV light. The light emitted by UV LED arrays 115A, 115B radiates from an inner surface of housing 110 in an angled and/or downward direction toward fingerprint scanner surface 151.

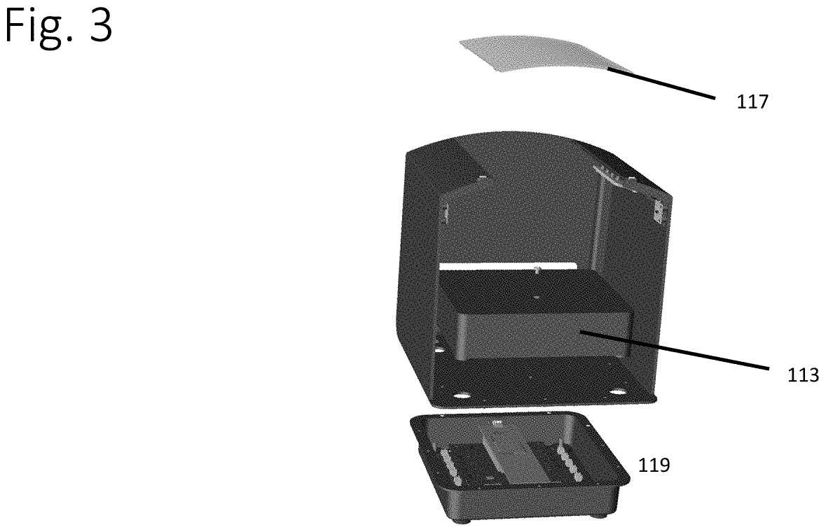

FIG. 3 illustrates an expanded perspective view of a sanitized fingerprint scanner 100 according to an example embodiment of the present invention. As shown in FIG. 3, sanitized fingerprint scanner 100 includes an adaptor 113, cover 117, and circuit cabinet 119.

Cover 117 is composed of a lightweight and transparent or quasi-transparent material, such as polymethyl methacrylate (PMMA). In some configurations, cover 117 is optional. Cover 117 is used to ensure that there is one possible space to insert one or more fingers of a user such that the user's presence can be easily detected by sensors 130A, 130B

Adaptor 113 can be composed of the same material as housing 110, and be configured to have a variable or adjustable height to accommodate different commercially available fingerprint scanners. For example, adaptor 113 could be split into a plurality of components the along the horizontal direction, to include multiple adapter components to adjust the adaptor height.

Circuit cabinet 119 is configured to enclose the electronics that control components of the sanitized fingerprint scanner 100, including UV LED arrays 115A, 115B, signaling lights 120A, 120B, sensors 130A, 130B, and fingerprint scanner 150. A combination of software (e.g., software that integrates into different software development kits (SDKs)) and hardware can be used. Circuit cabinet 119 is configured to be easily interchangeable, and can be couple to housing 110 using a variety of connector types such as screw, slide, tongue, and groove, etc. Power can be provided via either USB (e.g., USB 3.0) or a power supply from wall socket.

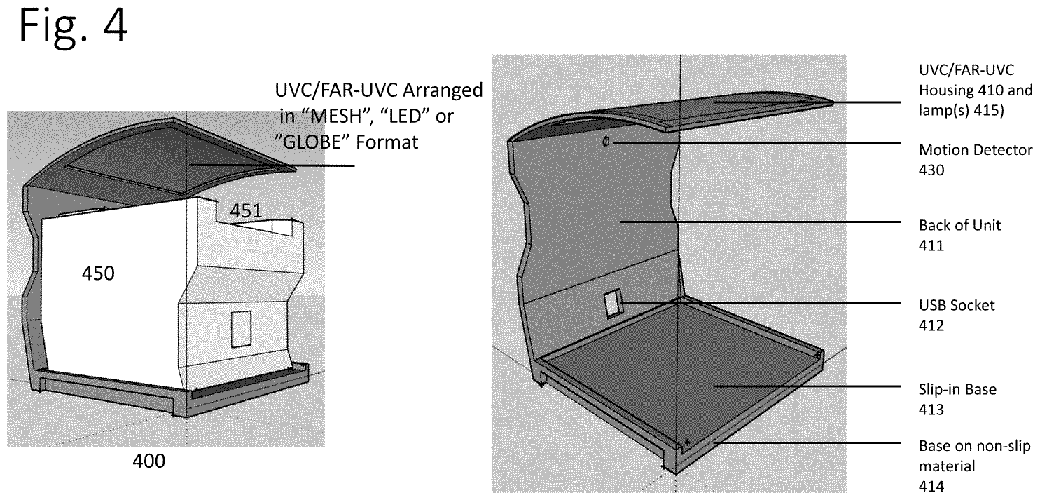

FIG. 4 illustrates a perspective front view of a sanitized fingerprint scanner 400 according to another example embodiment of the present invention. As shown in FIG. 4, sanitized fingerprint scanner 400 includes housing 410, sensor 430, fingerprint scanner 450, and the fingerprint scanner surface 451.

Housing 410 is configured to structurally support the components of the sanitized fingerprint scanner 400 including UV lights 415, sensor 430, fingerprint scanner 450, etc. Housing 410 can be formed from various lightweight materials. Example materials include a variety of plastics, preferably durable and lightweight plastics (e.g., acrylonitrile butadiene styrene (ABS), poly carbonate (PC), polyvinyl chloride (PVC), or combination thereof). Lightweight metals, such as aluminum, also can be used. Housing 110 can be formed by a molding, injection molding, three-dimensional (3D) printing process, etc.

Although not shown in FIG. 4, one or more signaling lights can be used, similar to signaling lights 120. Low-power LED lights configured to signal and instruct a user to input one or more fingers on the fingerprint scanner surface 451 or to remain clear of the sanitized fingerprint scanner 400 during a UV sanitization timeperiod or process.

Sensor 430 is configured to detect whether one or more fingers of a user accesses fingerprint contact surface 451. Sensor 430 can be disposed around the fingerprint contact surface 451 or along back surface 411 to detect whether one or more fingers of a user or other object approaches or covers the fingerprint contact surface 451.

Sensor 430 detects motion to determine if a finger(s) are on fingerprint contact surface 451 or under the UV light, to prevent exposure of UV light to skin and eyes. Sensor 430 is configured to halt or terminate any ongoing UV sanitizing process upon detection of a user's hand, finger, or other object. Other sensors (not shown) can be used to disable the UV sanitizing process when a user is within close proximity (e.g., 3-4 feet) to sanitized fingerprint scanner 400.

Slip-in base 413 is an opening on the front side of sanitized fingerprint scanner 400. The width of slip-in base 413 can be varied to accommodate a variety of fingerprint scanners, such as single-fingerprint scanners, dual-fingerprint scanners, multi-fingerprint scanners (e.g., 4-4-2 fingerprint sensors/readers), etc. In addition, a non-skid material can be applied on either the surface of slip-in base 413 or the bottom surface of housing 410.

As discussed above, a fingerprint scanner is described as an example embodiment, but it should be understood that the embodiments can be readily applied to a variety of device types.

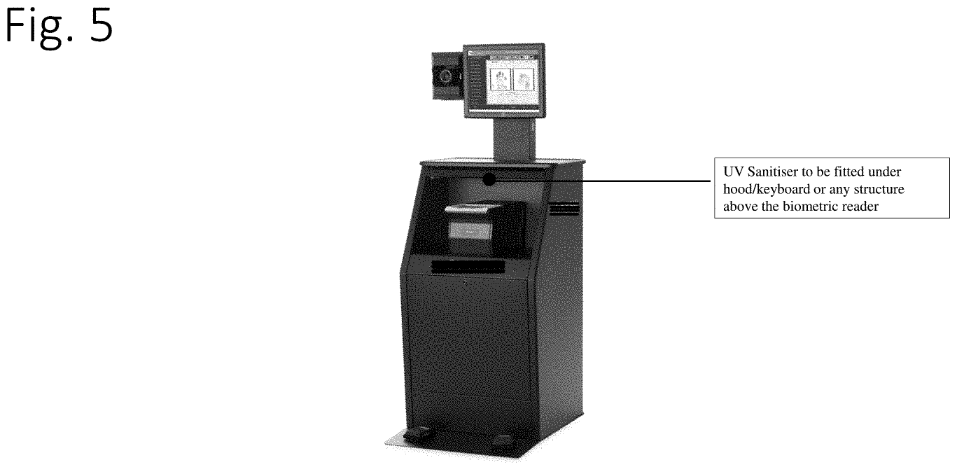

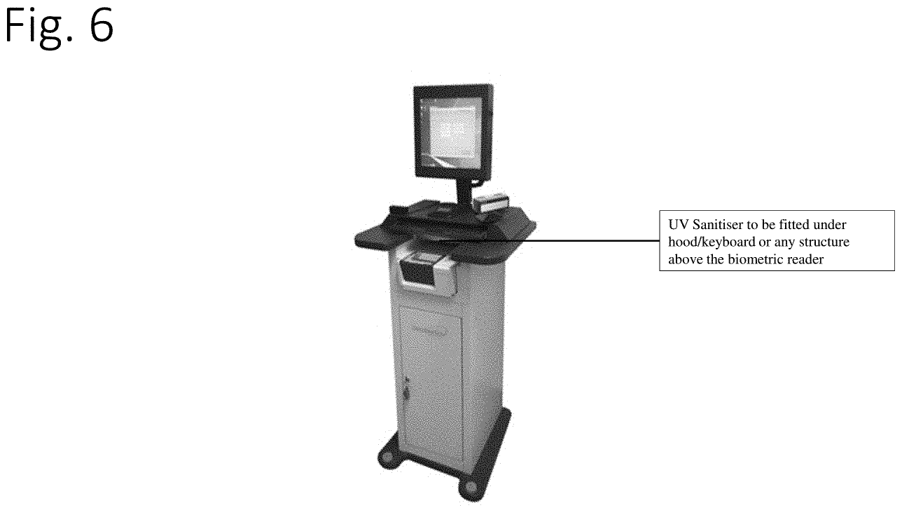

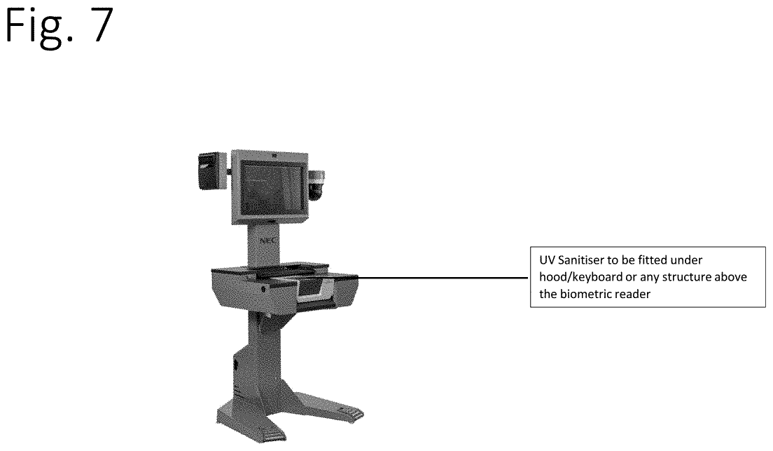

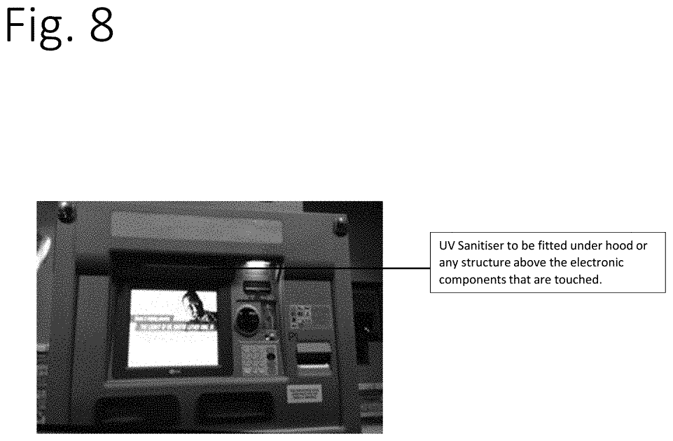

FIGS. 5, 6, 7, and 8 each illustrate a sanitized kiosk according to example embodiments of the present invention. In each of FIGS. 5, 6, 7, and 8, one or more UV lights are incorporated into a kiosk (e.g., point of servicer device, self-service terminal, ATM, etc.) that includes a variety of other electronic components (e.g., fingerprint sensors, cash dispensers, keypads, document scanners, signature pads, cameras, etc.) used to biometrically register/authenticate a user's identity in order to complete a transaction. The one or more UV lights are configured to radiate over frequently touched user-interface components to ensure their cleanliness.

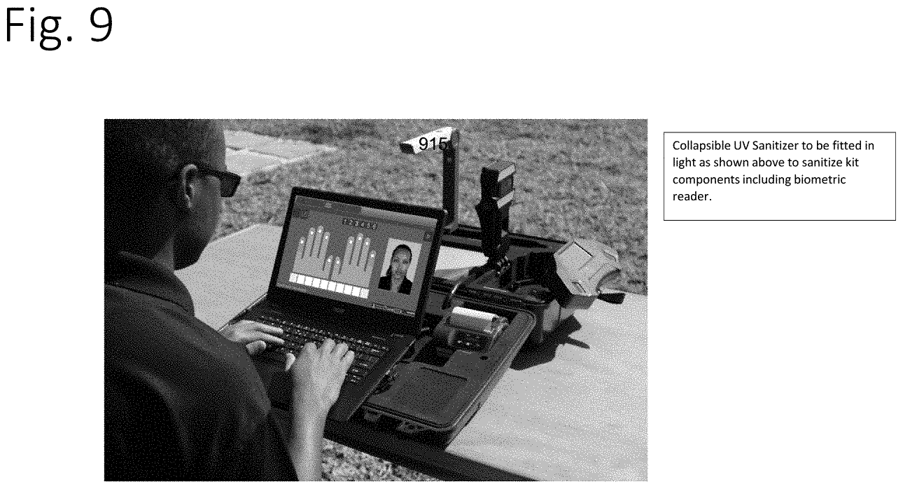

FIG. 9 illustrate a sanitized mobile computing device according to an example embodiment of the present invention. The UV sanitization lights 915 can be coupled to or otherwise integrated into a mobile biometric kit/jump-kit that is integrated with various electronic components (fingerprint sensors, signature pads, document scanners, cameras, card readers, laptops, etc.) used to biometrically register/authenticate a persons identity in order to complete a transaction. The one or more UV lights 915 are configured to radiate over frequently touched user-interface components to ensure their cleanliness.

As discussed above, a retrofit or integrated, automatic UV light biometric fingerprint sanitizing solution or other sanitized electronic device is provided by the example embodiments. The embodiments provide automatic and effective sanitization against a range of fungi, bacteria, viruses, and other contagions that can be found on biometric fingerprint readers and/or other input output devices to render it safe for continuous use by different people in a short period of time without the need of detergent sanitization products. The embodiment offer follow-on users peace of mind against contracting a pathogenic fungus, bacteria, virus, or other contagion from using a biometric fingerprint reader. The embodiments of the present invention can retrofitted to existing fingerprint scanners. Alternatively, the embodiments of the present invention can be incorporated into the design and manufacture of new fingerprint scanners. Although example configurations have been described, alternative configurations are feasible. For example, a sanitized fingerprint scanner may comprise a crane-like structure including a mast portion that is physically coupled to a jib portion. In this example configuration, a sanitizing unit including one or more sensors and one or more UV light arrays may be placed in proximity of an existing fingerprint scanner. Here, the sanitizing unit may be adjusted, raised, or lowered from the jib or the height of the mast may be adjusted, raised, or lowered.

In the various embodiments, the systems, devices, and methods for sanitizing a fingerprint scanner include a sanitized fingerprint scanner comprising a housing, one or more signaling lights, one or more sensors, one or more UV light arrays, and a fingerprint scanner having a fingerprint scanner surface, the sanitized fingerprint scanner configured to: obtain one or more fingerprints of a first user at the fingerprint scanner surface; after obtaining the one or more fingerprints of the first user, enabling the one or more UV light arrays to radiate light toward the fingerprint scanner surface, and after radiating the UV light toward the fingerprint scanner surface for a predetermined period of time, authorizing a second user to use the fingerprint scanner.

It will be apparent to those skilled in the art that various modifications and variations can be made in the systems, devices, and methods for sanitizing a fingerprint scanner of the present invention without departing from the spirit or scope of the present invention. Thus, it is intended that the present invention cover the modifications and variations of this invention provided they come within the scope of the appended claims and their equivalents.

We claim:

1. A sanitized fingerprint scanner comprising:
a housing;
one or more signaling lights to instruct one or more users;
one or more sensors to detect the presence of the one or more users;
one or more ultraviolet light arrays; and
a fingerprint scanner having a fingerprint scanner surface, the sanitized fingerprint scanner configured to:
obtain one or more fingerprints of a first user at the fingerprint scanner surface;
after obtaining the one or more fingerprints of the first user, enabling the one or more ultraviolet light arrays to radiate light toward the fingerprint scanner surface, and
after radiating the ultraviolet light toward the fingerprint scanner surface for a predetermined period of time, authorizing a second user to use the fingerprint scanner.

2. The sanitized fingerprint scanner according to claim 1, wherein the one or more sensors are configured to halt an ongoing ultraviolet sanitizing process upon detection of a user's hand, finger, or other object.

3. The sanitized fingerprint scanner according to claim 1, wherein the one or more ultraviolet light arrays are configured to emit ultraviolet light including UVA (315-400 nm), UVB (280-315 nm), UVC (100-280 nm, including far-UVC 207-222 nm), or a combination thereof.

4. The sanitized fingerprint scanner according to claim 1, wherein each bulb of the one or more ultraviolet light arrays is configured to emit multiple types of ultraviolet light.

5. The sanitized fingerprint scanner according to claim 1, wherein power is supplied to the sanitized fingerprint scanner by a USB port.

7. The sanitized fingerprint scanner according to claim 1, wherein housing is formed by a molding, injection molding, or three-dimensional (3D) printing process.

8. The sanitized fingerprint scanner according to claim 1, wherein housing is formed of acrylonitrile butadiene styrene (ABS), poly carbonate (PC), polyvinyl chloride (PVC), or a combination thereof.

9. The sanitized fingerprint scanner according to claim 1, wherein housing has a crane-like structure.

10. The sanitized fingerprint scanner according to claim 1, wherein the sanitized fingerprint scanner has a slip-in base configured to receive the fingerprint scanner.

11. The sanitized fingerprint scanner according to claim 1, wherein the sanitized fingerprint scanner has an adjustable base configured to receive the fingerprint scanner.
